(19) United States
(12) Patent Application Publication
(10) Pub. No.: US 2025/0248818 A1
(43) Pub. Date: Aug. 7, 2025

(54) EXTENDABLE LEAFLET-GRASPING DEVICE

(71) Applicant: EDWARDS LIFESCIENCES CORPORATION, Irvine, CA (US)

(72) Inventors: Yaron Herman, Givat Ada (IL); Ofry Efraim Yossef, Tel-Aviv (IL)

(21) Appl. No.: 19/191,265

(22) Filed: Apr. 28, 2025

Related U.S. Application Data
(63) Continuation of application No. PCT/US2023/035959, filed on Oct. 26, 2023.
(60) Provisional application No. 63/420,512, filed on Oct. 28, 2022.

(51) Int. Cl. A61F 2/24; A61F 2/48

(52) U.S. Cl. CPC A61F 2/2466; A61F 2/2463; A61F 2/48; A61F 2220/0008

(57) ABSTRACT

A leaflet-grasping tool comprises a shaft having a distal portion that is transluminally advanceable to a valve of a heart. The leaflet-grasping tool further comprises a first wire, advanceable out of the distal portion such that the first wire extends laterally away from the shaft to form an upstream support, and a second wire, advanceable out of the distal portion such that the second wire extends laterally away from the shaft to form a downstream support. The leaflet-grasping tool further comprises a control mechanism, attached to the second wire and adapted to deflect the downstream support with respect to the shaft. Other implementations are also disclosed.

### CROSS-REFERENCES TO RELATED APPLICATIONS

The present application is a continuation of International Patent Application No. PCT/US2023/035959, filed Oct. 26, 2023, which claims the benefit of U.S. Patent Application No. 63/420,512, filed Oct. 28, 2022, the entire disclosures all of which are incorporated by reference for all purposes.

### BACKGROUND

The native heart valves (i.e., the aortic, pulmonary, tricuspid and mitral valves) serve critical functions in assuring the forward flow of an adequate supply of blood through the cardiovascular system. These heart valves can be rendered less effective by congenital malformations, inflammatory processes, infectious conditions or disease. Valve regurgitation occurs when the native valve fails to close properly and blood flows into an atrium from a ventricle during the systole phase of heart contraction. Valve regurgitation is the most common form of valvular heart disease. Such damage to the valves can result in serious cardiovascular compromise or death.

Treatment for such disorders can be done with the surgical repair or replacement of the valve during open heart surgery or with transcatheter transvascular techniques for introducing and implanting prosthetic devices in a manner that is much less invasive than open heart surgery.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, it can be advantageous to securely grasp a leaflet of a valve of a real or simulated heart during repair and/or replacement of the valve. For example, during implantation of a leaflet-anchored implant, such as an artificial chorda tendinea and/or a leaflet-augmentation patch, it can be desirable to securely grasp the leaflet prior to anchoring the implant to the leaflet, e.g., in order to stabilize the leaflet during the anchoring.

In some implementations, a leaflet-grasping tool includes a shaft (e.g., a catheter, tube, lumen, etc.) having a distal portion that is configured to be transluminally advanceable to a valve of the heart (e.g., its material properties, flexibility, stiffness, hardness, surface properties, cut pattern, etc. are sufficient to allow transluminal or transvascular advancement). In some implementations, the shaft is a component of a delivery assembly (e.g., a delivery tool thereof) that can be used for additional functions other than grasping the leaflet, e.g., to anchor another part of the implant within a ventricle downstream of the valve. In some implementations, the shaft is a component of an implant.

In some implementations, the leaflet-grasping tool can comprise a first wire, attached to the distal portion of the shaft, and advanceable away from (e.g., out of) the distal portion such that the first wire extends (e.g., arcs) laterally away from the shaft to form an upstream support. Similarly, the leaflet-grasping tool can comprise a second wire, attached to the distal portion of the shaft, and advanceable away from (e.g., out of) the distal portion such that the second wire extends (e.g., arcs) laterally away from the shaft to form a downstream support.

In some implementations, the leaflet-grasping tool is configured to grasp the leaflet by sandwiching the leaflet between the upstream support and the downstream support, e.g., such that the first wire presses against an upstream surface of the leaflet (e.g., an atrial-facing surface of the leaflet), and the second wire presses against a downstream surface of the leaflet (e.g., a ventricle-facing surface of the leaflet).

In some implementations, in order to grasp a portion of the leaflet that has a sufficiently large area, the first wire (e.g., the upstream support formed by the wire) can be advanced over the leaflet (e.g., over the upstream surface of the leaflet) while deflected away from the downstream support (and out of contact with the leaflet), prior to sandwiching the portion of the leaflet between the upstream support and the downstream support. That is, the upstream support and the downstream support can act in a similar manner to a pair of jaws, except that the length of one or both jaws is adjustable independently of opening and closing the jaws.

In some implementations, a system and/or apparatus (e.g., for use at a real or simulated heart, etc.) includes a leaflet-grasping tool (the leaflet-grasping tool can be the same as or similar to the above leaflet-grasping tool or other leaflet-grasping tools described elsewhere herein).

In some implementations, the leaflet-grasping tool can include a shaft having a distal portion that is transluminally advanceable to a valve of the heart.

In some implementations, the leaflet-grasping tool can include a wire (e.g., a first wire), advanceable out of the distal portion such that the first wire extends laterally away from the shaft to form an upstream support.

In some implementations, the leaflet-grasping tool can also comprise a second wire, advanceable out of the distal portion such that the second wire extends laterally away from the shaft to form a downstream support.

In some implementations, the leaflet-grasping tool comprises a control rod, attached to the first wire and adapted to deflect the upstream support with respect to the shaft.

In some implementations, the control rod is hingedly attached to the first wire.

In some implementations, the distal portion is transluminally advanceable to the valve such that the shaft extends, from the distal portion at the valve, through vasculature of the subject, and out of the subject.

In some implementations, the control rod is adapted to deflect the upstream support with respect to the shaft independently of advancing the first wire laterally away from the shaft.

In some implementations, the leaflet-grasping tool is sterile. The scope of the disclosure includes a method that includes sterilizing the leaflet-grasping tool.

In some implementations, the control rod is adapted to deflect the upstream support with respect to the shaft without increasing a lateral distance between the upstream support from the shaft.

In some implementations, the first wire defines a distal part that is adapted to contact an atrial surface of a leaflet of the valve, and the control rod is attached to the distal part of the first wire.

In some implementations, the leaflet-grasping tool is adapted to grasp a leaflet of the valve by sandwiching a portion of the leaflet between the upstream support and the downstream support by (i) pressing the upstream support against an atrial surface of the leaflet, and (ii) pressing the downstream support against a ventricular surface of the leaflet.

In some implementations, the leaflet-grasping tool is configured to dynamically limit lateral extension of the first wire out of the distal portion according to lateral extension of the second wire out of the distal portion.

In some implementations, the leaflet-grasping tool is configured to dynamically limit lateral extension of the second wire out of the distal portion according to lateral extension of the first wire out of the distal portion.

In some implementations, the leaflet-grasping tool includes a control mechanism (e.g., one or more of a rod, control rod, wire, tube, hypotube, line, suture, knob, slider, gear, motor, control, gimble, cam, etc.) adapted to deflect the downstream support with respect to the shaft.

In some implementations, the control mechanism is adapted to push distally on the downstream support in order to deflect the downstream support.

In some implementations, the control mechanism is configured to constrain the downstream support deflected during advancement of the second wire out of the distal portion.

In some implementations, the leaflet-grasping tool is adapted to grasp the leaflet with the upstream support and the downstream support flat against the atrial surface and the ventricular surface, respectively.

In some implementations, the first wire defines a frame, and the upstream support includes a sheet attached to the frame, such that the sheet presses against the atrial surface of the leaflet.

In some implementations, the second wire defines a frame, and the downstream support includes a sheet attached to the frame, such that the sheet presses against the ventricular surface of the leaflet.

In some implementations, the control rod is configured to hold the upstream support deflected away from the leaflet during advancement of the first wire out of the distal portion.

In some implementations, the control rod is configured to facilitate the sandwiching by deflecting the upstream support toward the downstream support.

In some implementations, the system/apparatus includes an implant, and the leaflet-grasping tool is adapted to secure the implant to the leaflet while grasping the leaflet.

In some implementations, the implant includes an anchor that is adapted to anchor the implant to the leaflet, the control rod defines a channel adapted to house the anchor therewithin, and the leaflet-grasping tool is adapted to advance the anchor out of the channel and into the leaflet.

In some implementations, a bight of the second wire is arranged in a loop, the downstream support including the loop of the second wire, and the leaflet-grasping tool is adapted to advance the anchor out of the channel and into a portion of the leaflet that is delineated by the loop of the second wire.

In some implementations, the anchor includes a toggle and a tether extending away from the toggle, and the anchor is advanceable through the leaflet and into a ventricle downstream of the leaflet, such that the tether extends, from the toggle in the ventricle, through the leaflet, and into an atrium upstream of the leaflet.

In some implementations, the control rod is attached to the first wire such that pressing the upstream support against the atrial surface positions a distal opening of the channel against the leaflet.

In some implementations, the leaflet-grasping tool includes a needle, extending through the channel and housing the anchor, and the anchor is advanceable out of the channel and into the leaflet by advancing the needle out of the channel and into the leaflet.

In some implementations, the leaflet-grasping tool includes a pusher, adapted to push the anchor out of the channel and towards the leaflet.

In some implementations, the leaflet-grasping tool is withdrawable from the heart subsequently to securing the implant to the leaflet, by: (i) withdrawing the first wire from the leaflet and back towards the shaft, (ii) withdrawing the second wire from the leaflet and back towards the shaft, and (iii) withdrawing the shaft from the heart.

In some implementations, a bight of the first wire is arranged in a loop, the upstream support including the loop.

In some implementations, the control rod is attached to the loop.

In some implementations, the control rod is hingedly attached to the first wire by being attached to a cuff through which the loop extends.

In some implementations, the first wire has a first stretch and a second stretch that each extend along at least part of the shaft, the bight connecting the first stretch to the second stretch at the distal portion.

In some implementations, the leaflet-grasping tool has a retracted state in which the loop is disposed against an external surface of the distal portion of the shaft.

In some implementations, a bight of the second wire is arranged in a loop, the downstream support including the loop of the second wire.

In some implementations, the second wire has a first stretch and a second stretch that each extend along at least part of the shaft, the bight of the second wire connecting the first stretch of the second wire to the second stretch of the second wire at the distal portion.

In some implementations, the leaflet-grasping tool has a retracted state in which the loop is disposed against an external surface of the distal portion of the shaft.

In some implementations, the leaflet-grasping tool is configured such that advancement of the first wire out of the distal portion enlarges the loop.

In some implementations, the first wire is advanceable out of the distal portion by increasing a length of the first wire exposed within the heart.

In some implementations, the first wire is slidably coupled to the shaft. In some implementations, the first wire is advanceable out of the distal portion by sliding the first wire distally away from the shaft.

In some implementations, the control rod is attached to the upstream support.

In some implementations, the control rod is attached to a tip of the upstream support.

In some implementations, the leaflet-grasping tool includes a controller, coupled to a proximal portion of the shaft, the controller adapted to adjust the lateral extension of the first wire away from the shaft.

In some implementations, the controller is adapted to adjust the lateral extension of the first wire away from the shaft between a plurality of discrete extensions.

In some implementations, the controller is adapted to adjust the lateral extension of the first wire away from the shaft over a continuum of extensions.

In some implementations, the controller is adapted to control the deflection of the upstream support with respect to the shaft, independently of the lateral extension of the first wire away from the shaft.

In some implementations, the controller is adapted to adjust the lateral extension of the second wire away from the shaft independently of the lateral extension of the first wire away from the shaft.

In some implementations, the control rod is a first control rod. In some implementations, the system/apparatus includes a second control rod that is attached to the first wire. In some implementations, the second control rod is adapted to deflect the upstream support with respect to the shaft.

In some implementations, the first control rod is operable independently of the second control rod.

In accordance with some implementations, a system and/or an apparatus (e.g., a real or simulated includes a leaflet-grasping tool. The leaflet-grasping tool can be the same as or similar to the leaflet-grasping tools described above or elsewhere herein.

In some implementations, the leaflet-grasping tool includes a shaft, having a distal portion that is transluminally advanceable to a valve of the heart.

In some implementations, the leaflet-grasping tool includes a first wire, advanceable out of the distal portion such that the first wire extends laterally away from the shaft to form an upstream support.

In some implementations, the leaflet-grasping tool comprises a second wire, advanceable out of the distal portion such that the second wire extends laterally away from the shaft to form a downstream support.

In some implementations, the leaflet-grasping tool comprises a control rod, adapted to deflect, with respect to the shaft, a support selected from the group consisting of the upstream support and the downstream support, independently of lateral extension of the wire of the selected support away from the shaft.

In some implementations, the selected support is the upstream support.

In some implementations, the selected support is the downstream support.

In accordance with some implementations, a method (e.g., for use at a heart, such as a living heart or non-living simulation) includes transluminally advancing a distal portion of a shaft of a leaflet-grasping tool to a valve of the heart.

In some implementations, the method includes forming a downstream support by advancing a downstream wire of the leaflet-grasping tool out of the distal portion and laterally away from the shaft.

In some implementations, the method includes forming an upstream support by advancing an upstream wire of the leaflet-grasping tool out of the distal portion and laterally away from the shaft.

In some implementations, the method includes placing the downstream support against a downstream surface of a leaflet of the valve.

In some implementations, the method includes placing the upstream support against an upstream surface of the leaflet by deflecting the upstream support with respect to the shaft and toward the downstream support.

In some implementations, the method includes, prior to placing the upstream support against the upstream surface, deflecting the upstream support with respect to the shaft and away from the downstream support.

In some implementations, the method includes, while the upstream support remains against the upstream surface, and the downstream support remains against the downstream surface, advancing an anchor into the leaflet.

In some implementations, advancing the anchor into the leaflet includes driving the anchor out of the leaflet-grasping tool and into the leaflet.

In some implementations, advancing the anchor into the leaflet includes advancing the anchor into a portion of the leaflet delineated by the downstream support.

In some implementations, the method further includes sterilizing the leaflet-grasping tool.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present invention will be more fully understood from the following detailed description of implementations thereof, taken together with the drawings.

### BRIEF DESCRIPTION OF THE DRAWINGS

FIGS. 1A-C are schematic illustrations of a system adapted to grasp a leaflet of a valve, in accordance with some implementations; and

FIGS. 2A-M are schematic illustrations of a series of steps for use with the system, in accordance with some implementations.

### DETAILED DESCRIPTION

In some implementations, it may be advantageous to securely grasp a leaflet of a valve of the heart during repair and/or replacement of the valve. For example, during implantation of a leaflet-anchored implant, such as an artificial chorda tendinea and/or a leaflet-augmentation patch, it may be desirable to securely grasp the leaflet prior to anchoring the implant to the leaflet, e.g., in order to stabilize the leaflet during the anchoring.

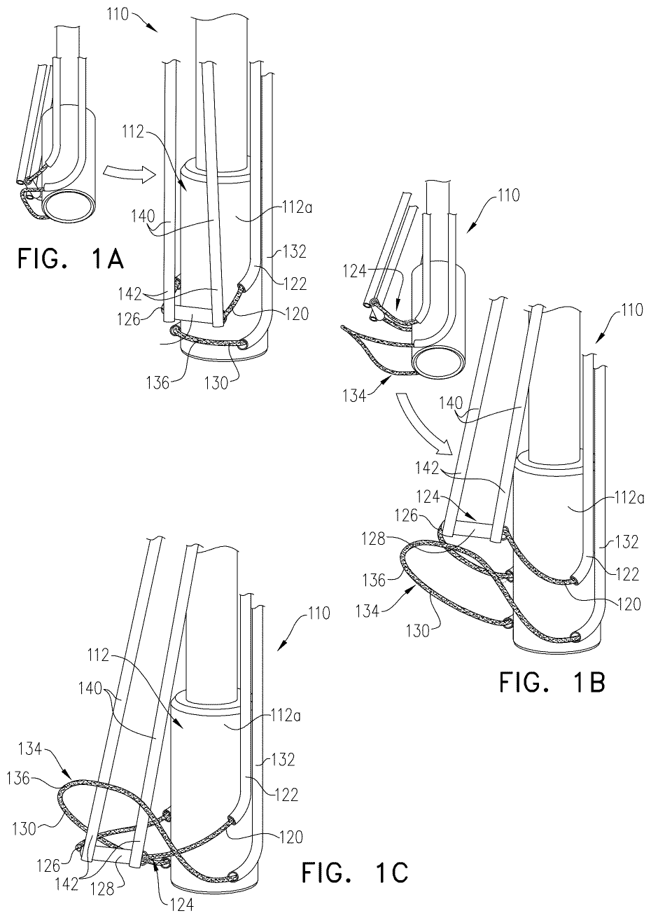

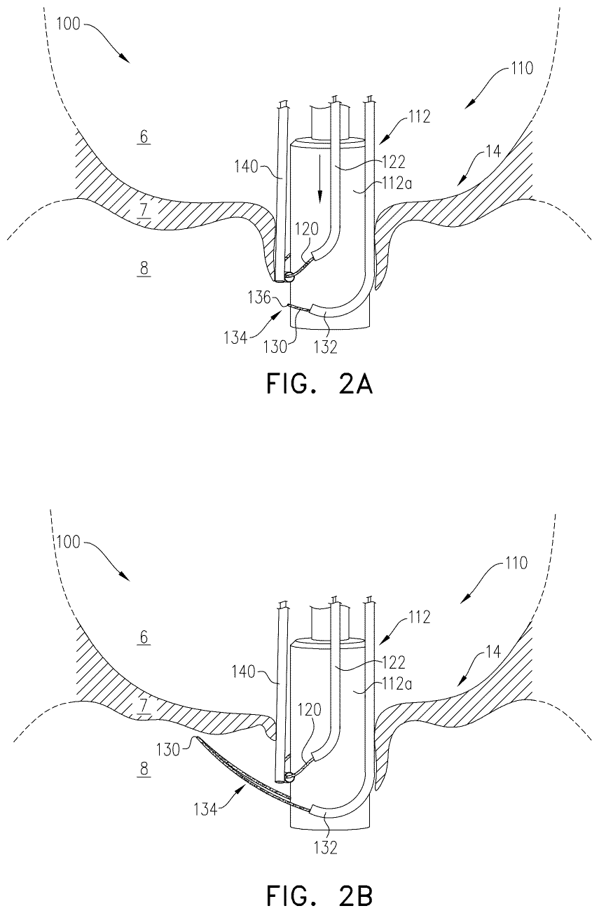

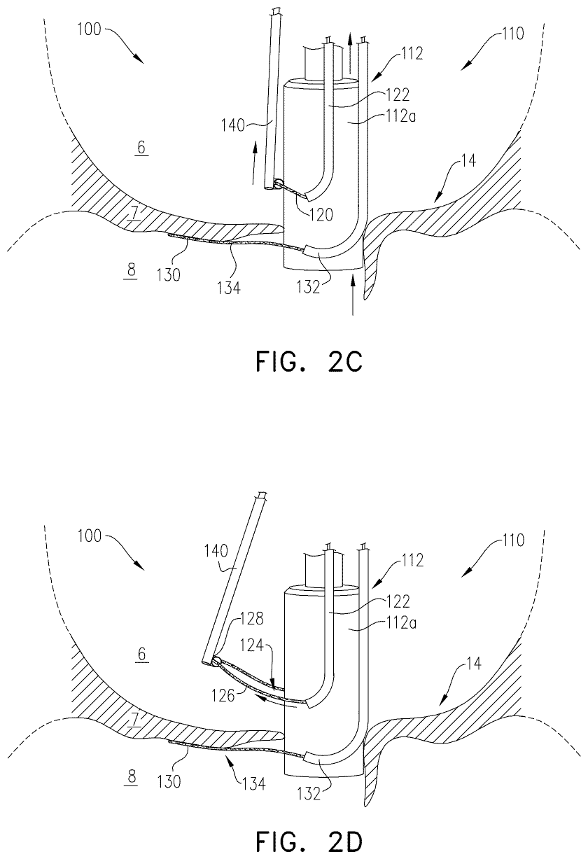

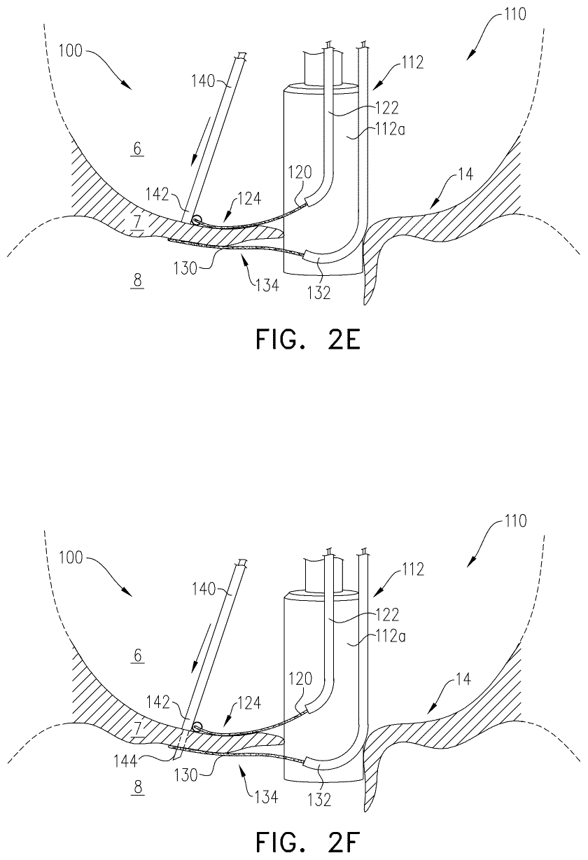

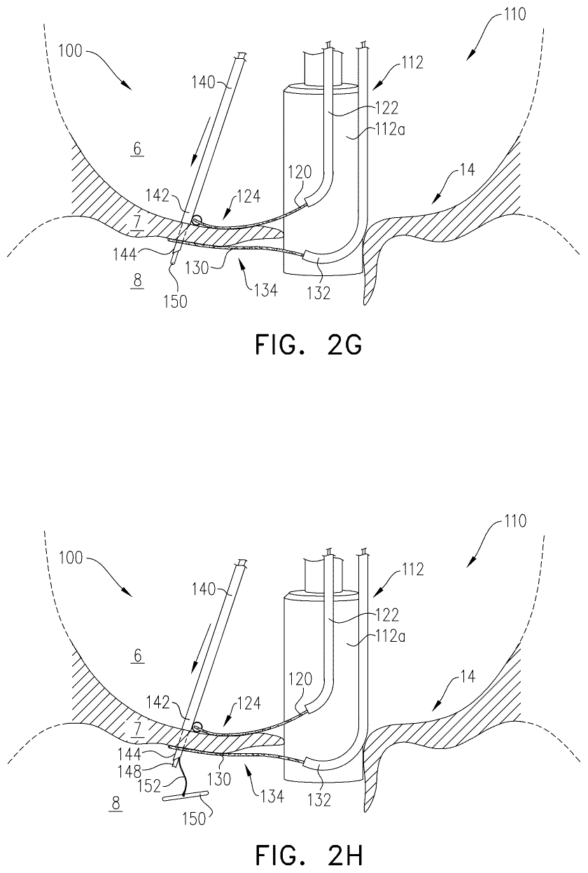

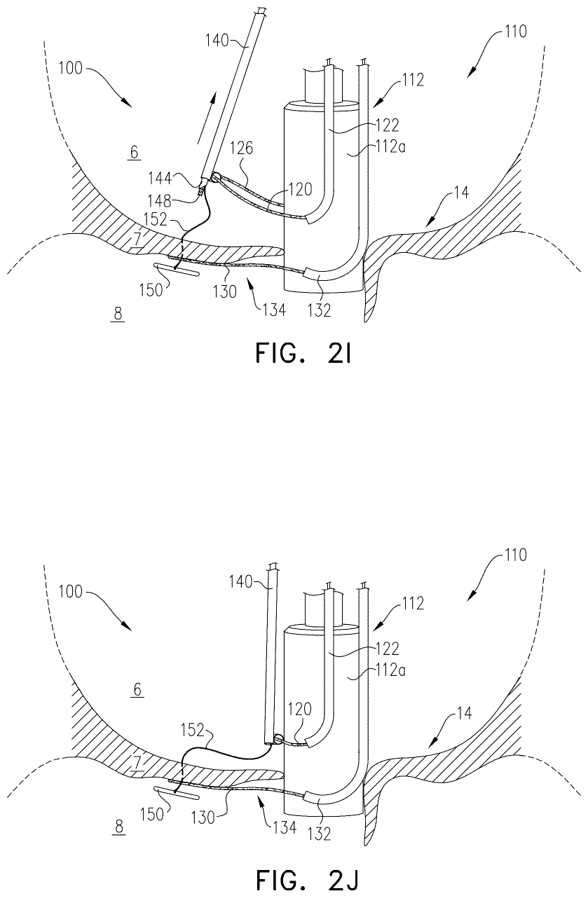

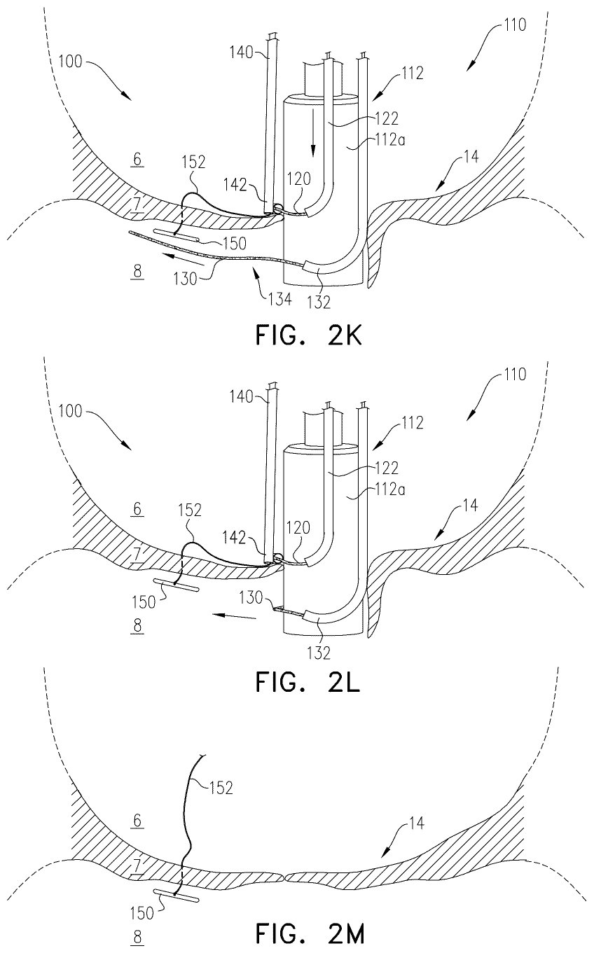

Reference is made to FIGS. 1A-C, and 2A-M, which are schematic illustrations of a system 100 adapted to grasp a leaflet 7 of a valve 14, in accordance with some implementations. FIGS. 1A-C show a perspective view of system 100. FIGS. 2A-M show a series of steps that can be used with system 100.

System 100 comprises a leaflet-grasping tool 110 including a shaft 112 (e.g., a catheter, tube, sheath, or rod), having a distal portion 112a that is transluminally advanceable to valve 14 of the heart.

Leaflet-grasping tool 110 comprises a first wire 120, attached to distal portion 112a of shaft 112, and advanceable away from (e.g., out of) the distal portion such that the first wire extends (e.g., arcs) laterally away from the shaft to form an upstream support 124. Similarly, leaflet-grasping tool 110 comprises a second wire 130, attached to distal portion 112a of shaft 112, and advanceable away from (e.g., out of) the distal portion such that the second wire extends (e.g., arcs) laterally away from the shaft to form a downstream support 134.

In some implementations, leaflet-grasping tool 110 is reversibly transitionable from a retracted state (e.g., as shown in FIGS. 1A and 2A) in which the first wire and the second wire are disposed against an external surface of the distal portion of shaft, to a state in which both wires extend laterally away from the distal portion, thus forming the upstream and downstream supports respectively (e.g., as shown in FIGS. 1C and 2E).

In some implementations, during use of tool 110 (e.g., while distal portion 112a is disposed within the heart) advancing the first wire out of distal portion 112a and away from shaft 112 increases the amount of the wire exposed within the heart (e.g., exposed away from the shaft). In some implementations, the first wire extends, from an extracorporeal handle of leaflet-grasping tool 110 (e.g., at a proximal portion of tool 110), along the shaft (e.g., along a lumen thereof, or along an outside wall of the shaft, e.g., within an external channel 122 that is attached to the shaft), to distal portion 112a.

In some implementations, first wire 120 is advanceable away from the shaft by pushing the wire distally from outside the subject, e.g., by actuating a controller (not shown) on the handle of tool 110. For example, actuating the controller can adjust the extent (e.g., the effective area) of the upstream support by adjusting the lateral extension of the first wire away from the shaft.

In some implementations, the controller is adapted to transition the first wire between a plurality of extents of lateral extension (e.g., discrete and/or preset size settings), or over a continuum of extents of lateral extension (e.g., without discrete and/or preset size settings). In some implementations, first wire 120 is slidably coupled to shaft 112 (e.g., to an external surface of the shaft), such that the first wire is advanceable out of distal portion 112a by sliding the first wire distally away from the shaft.

In some implementations, first wire 120 defines a loop 126, e.g., a bight of first wire 120 is arranged in the loop, such that upstream support 124 comprises the loop. In some implementations, a first stretch of wire 120 extends along the shaft (e.g., from the extracorporeal handle) to distal portion 112a, where its bight defines loop 126, and back along the shaft in a second stretch (e.g., back along the shaft to the extracorporeal handle). In some implementations, advancing first wire 120 out of distal portion 112a enlarges loop 126, thereby forming the upstream support.

In some implementations, and similarly to as described hereinabove with reference to first wire 120, advancing second wire 130 out of distal portion 112a and away from shaft 112 increases the amount of the wire exposed within the heart (e.g., exposed away from the shaft).

In some implementations, the second wire extends, from the extracorporeal handle, along shaft 112 (e.g., along a lumen thereof, or along an outside wall of the shaft, e.g., within an external channel 132 that is attached to the shaft), to distal portion 112a.

In some implementations, second wire 130 is advanceable away from the shaft by pushing the wire distally from outside the subject, e.g., by actuating a controller on the handle of tool 110. In some implementations, second wire 130 is slidably coupled to shaft 112 (e.g., to an external surface of the shaft), such that the second wire is advanceable out of distal portion 112a by sliding the second wire distally away from the shaft.

In some implementations, and as described hereinabove with reference to first wire 120, second wire 130 defines a loop 136, e.g., a bight of second wire 130 is arranged in the loop, such that downstream support 134 comprises the loop. In some implementations, a first stretch of the first wire extends along the shaft (e.g., from the extracorporeal handle) to the distal portion, where the bight of loop 136 is defined, and back along the shaft in a second stretch (e.g., back along the shaft to the extracorporeal handle). In some implementations, advancing second wire 130 out of distal portion 112a enlarges loop 136, thereby forming the downstream support.

Although upstream support 124 and downstream support 134 are each shown as consisting essentially of a loop of their respective wire, in some implementations, the upstream support and/or the downstream support can comprise one or more additional components. Such additional components can also be defined by the wire (e.g., the wire can be shaped such that the loop is somewhat filled in by the wire), e.g., by the wire having a shape such as a serpentine or spiral shape. Alternatively, or additionally, such additional components can be attached to the wire, e.g., a sheet (e.g., a fabric or a film) or a mesh.

In some implementations, only one of upstream support 124 or the downstream support 134 defines an extendable support (e.g., a loop that is laterally extendable away from the shaft), and the other support is provided as a flap that is operated primarily (e.g., exclusively) by deflection, e.g., having a fixed length.

In some implementations, and as shown in FIGS. 2E-H, leaflet-grasping tool 110 is configured to grasp leaflet 7 by sandwiching the leaflet between upstream support 124 and downstream support 134, e.g., such that first wire 120 presses against an upstream surface of the leaflet (e.g., an atrial-facing surface of the leaflet), and second wire 130 presses against a downstream surface of the leaflet (e.g., a ventricle-facing surface of the leaflet). This can be done in order to stabilize the leaflet, e.g., during anchoring of a leaflet anchor to the leaflet.

In order to grasp a portion of the leaflet that has a sufficiently large area, first wire 120 (e.g., upstream support 124 formed by the wire) can be advanced over leaflet 7 (e.g., over the upstream surface of the leaflet) while deflected away from downstream support 134 (and out of contact with the leaflet), prior to sandwiching the portion of the leaflet between the upstream support and the downstream support. Additionally, or alternatively, in some implementations, second wire 130 can be advanced over leaflet 7 (e.g., over the downstream surface of the leaflet) while deflected away from upstream support 124 (and out of contact with the leaflet), prior to sandwiching the portion of the leaflet between the upstream support and the downstream support. That is, the upstream support and the downstream support may act in a similar manner to a pair of jaws, except that the length of one or both jaws is adjustable independently of opening and closing the jaws.

In some implementations, in order to deflect upstream support 124 in this manner (e.g., to facilitate the advancement of first wire 120 over leaflet 7), leaflet-grasping tool 110 comprises a control rod 140 that is adapted to deflect (e.g., to orient) upstream support 124 with respect to shaft 112 and/or with respect to downstream support 134.

In some implementations, pulling the control rod proximally deflects upstream support 124 upwardly (e.g., into an atrium 6 that is upstream from valve 14). For example, FIGS. 1B and 2D show control rod 140 holding (e.g., restraining) the upstream support in a deflected state.

In some implementations, control rod 140 is attached to first wire 120 (e.g., to loop 126 thereof). In some implementations, control rod 140 is attached to a distal part of first wire 120, e.g., to part of the wire that defines a distal tip of upstream support 124 thereof.

As described hereinabove, the control rod can be adapted or configured to deflect the upstream support with respect to the shaft independently of advancing the first wire laterally away from the shaft. For example, the controller can be adapted to provide independence between control of (i) the lateral extension of the upstream support, (ii) the lateral deflection of the downstream support, and (iii) deflection of the upstream support via the control rod, respectively, e.g., such that the control rod can deflect the upstream support with respect to the shaft without increasing an effective size of the upstream support.

In the example shown, control rod 140 is hingedly attached to first wire 120, e.g., by being fixedly attached to a cuff 128 through which loop 126 loops. This can facilitate deflection of upstream support 124 by allowing the upstream support to pivot with respect to control rod 140.

In some implementations, leaflet-grasping tool 110 comprises at least two control rods 140, each control rod being attached to first wire 120 (e.g., to loop 126 thereof) and adapted to deflect the upstream support with respect to the downstream support. In some implementations, each control rod is operable independently of the other control rod, e.g., such that different regions of upstream support 124 are deflectable with respect to each other (e.g., are deflectable at least partly independently of each other).

In some implementations, downstream support 134 is deflectable with respect to shaft 112 and/or to upstream support 124, e.g., in a similar manner to that described with reference to upstream support 124. For example, downstream support 134 can be deflectable via a control mechanism (e.g., one or more of a rod, control rod, wire, tube, hypotube, line, suture, knob, slider, gear, motor, control, gimble, cam, etc.). In some implementations, the control mechanism can include and/or be similar to control rod 140. In some implementations, the downstream support 134 can be deflectable via a bearing surface that pushes distally on the downstream support, e.g., close to a “root” of the downstream support where wire 130 leaves shaft 112.

In some implementations, in addition to grasping leaflet 7 (e.g., to stabilize the leaflet), leaflet-grasping tool 110 can also be used to anchor an anchor 150 of an implant to the leaflet. In some implementations, and as shown, control rod 140 (or at least a distal end of the control rod) defines a channel 142, and anchor 150 is housed within and/or advanced through the channel during delivery and/or anchoring.

In some implementations, the attachment of control rod 140 to first wire 120 such that placing the control rod against the leaflet positions the anchor channel close to (e.g., against) the leaflet, e.g., such that a distal opening of the anchor channel faces the atrial surface of the leaflet.

In some implementations in which multiple control rods 140 are used, each control rod can define an anchor channel that is adapted to house a respective anchor, e.g., the leaflet-grasping tool being adapted to anchor multiple anchors to leaflet 7.

In some implementations, once the leaflet is grasped by leaflet-grasping tool 110 (e.g., once the leaflet is sandwiched between upstream support 124 and downstream support 134), the anchor is advanced out of the anchor channel and into and/or through the leaflet.

In some implementations, anchor 150 is a toggle anchor, and can be advanceable through the leaflet such that the anchor becomes positioned within ventricle 8, e.g., such that the anchor lies against the ventricular surface of the leaflet.

In some implementations, enlargement of upstream support 124 is dynamically limited to the size of the downstream support, in order to ensure that anchor 150 is driven through the leaflet at an appropriate position with respect to the downstream support (e.g., within the area delineated by the downstream support) e.g., in order to provide a sufficient reference force against the leaflet during anchoring of the anchor. This dynamic limitation of the upstream support with respect to the downstream support can be provided at a handle of tool 110, e.g., via operative interaction between a controller (e.g., a control knob and/or a control slider) of the first wire and a controller of the second wire.

FIGS. 2A-M represent a series of steps that can be performed by the operator, to grasp leaflet 7 of valve 14, and optionally to anchor tissue anchors of an implant to the leaflet while the leaflet is being grasped, in accordance with some implementations. Although FIGS. 2A-M show a sequence of at least some steps in a procedure, and can, in fact, be performed in the order shown, these figures are also intended to illustrate the capability of system 100, independently of any particular sequence of steps. For example, although FIGS. 2A-M show downstream support being positioned against the leaflet prior to the positioning of upstream support 124 against the leaflet, it is to be understood that both supports could be positioned concurrently against the leaflet, or the upstream support could be positioned against the leaflet prior to the positioning of the downstream support against the leaflet.

FIG. 2A shows distal portion 112a of shaft 112 having been advanced to valve 14, such that loop 136 is disposed downstream of leaflet 7, e.g., in a ventricle 8 downstream of the valve. In order to form downstream support 134, the second wire is subsequently advanced out of distal portion 112a (e.g., away from shaft 112) such that the second wire extends laterally away from the shaft, e.g., over the downstream surface of leaflet 7 (FIG. 2B). Shaft 112 can subsequently be pulled proximally, causing the downstream support 134 to contact the downstream surface of the leaflet (FIG. 2C).

In order to sandwich the leaflet between upstream support 124 and downstream support 134, first wire 120 is then extended over, and placed in contact with, the upstream surface of leaflet 7, e.g., as shown in FIGS. 2D-E. FIGS. 2D-E show first wire 120 being advanced out of distal portion 112a (e.g., away from shaft 112) such that the first wire extends laterally away from the shaft.

In order to grasp a portion of the leaflet that has a sufficiently large area, first wire 120 (e.g., upstream support 124 formed by the wire) can be advanced out of distal portion 112a while deflected away from downstream support 134 (and out of contact with the leaflet), e.g., as shown in FIG. 2D. As described hereinabove, this can be achieved by pulling control rod 140 proximally, such that upstream support 124 is deflected upwards into atrium 6. In some implementations, this deflection can be performed concurrently and/or maintained with the extension of the upstream support. For example, FIG. 2D shows upstream support 124 being extended while being constrained in an upwardly deflected manner.

In some implementations, and as shown in FIG. 2C, first wire is deflected upwardly by pulling of control rod 140 prior to prior to extension of first wire 130 over the upstream surface of leaflet 7. For example, this can be performed prior to shaft 112 being pulled proximally towards atrium 6. This may allow leaflet 7 to become positioned between the two wires, e.g., by preventing first wire 130 and/or control rod 140 obstructing leaflet 7 from becoming optimally positioned between the two wires.

In some implementations, control rod 140 can be in such a retracted position during delivery, e.g., tool 110 can be provided with control rod 140 in this position.

In some implementations, upstream support 124 can be and/or is deflected downwardly such that it contacts the atrial surface of leaflet 7 (FIG. 2E), thereby sandwiching the leaflet between upstream support 124 and downstream support 134. In some implementations, this can be achieved simply by releasing control rod 140 (e.g., with wire 120 being biased to deflect upstream support 124 toward downstream support 134), and/or by the control rod actively pushing the upstream support toward the downstream support.

In some implementations, while leaflet 7 remains grasped by leaflet-grasping tool 110 (e.g., while the leaflet remains sandwiched between upstream support 124 and downstream support 134), anchor 150 can then be anchored to the leaflet. In some implementations in which control rod 140 (e.g., anchor channel 142 thereof) houses the anchor, the anchor can be advanced out of the anchor channel, and into leaflet 7.

In some implementations, leaflet-grasping tool 110 comprises a needle 144, which is housed within control rod 140 (e.g., within anchor channel 142 thereof), and is adapted to house anchor 150, e.g., such that the control rod houses the anchor by housing the needle that houses the anchor. FIGS. 2F-H illustrate an example in which needle 144 is first advanced out of control rod 140 and through leaflet 7 (FIG. 2F), and a pusher 148 is then used to push the anchor out of needle 144, e.g., into leaflet 7 (e.g., through the leaflet and into ventricle 8). FIG. 2G shows anchor 150 emerging from needle 144, with part of the anchor exposed within ventricle 8.

Once anchor 150 is full deployed (FIG. 2H), needle 144 can be retracted back through the leaflet and into the control rod (FIG. 21). In some implementations, a tether 152 is attached to anchor 150 and, once the anchor is deployed, extends, from the ventricle where the tether is attached to the anchor, through the leaflet, and into atrium 6.

In some implementations, control rod 140 (e.g., channel 142 thereof) defines a lateral slit that extends vertically along the control rod, such that once the anchor has been deployed, tether 152 can exit the control rod by exiting the slit laterally. In some implementations, upstream support 124 (FIG. 2J) and downstream support (FIGS. 2K-L) are then retracted, leaving the anchor implanted at leaflet 7 (FIG. 2M).

In some implementations, and as shown, withdrawal of downstream support 134 is facilitated by moving the support away from the leaflet (e.g., by advancing distal portion 112a further into ventricle 8) and/or by temporarily further enlarging the support, so as to clear the support from anchor 150 (FIG. 2K).

Reference is again made to FIGS. 1A-C and 2A-M. In some implementations, shaft 112 and/or tool 110 is a component of a delivery assembly (e.g., a delivery tool thereof) (not shown) that can be used for additional functions other than grasping the leaflet and/or anchoring an anchor of an implant to the leaflet.

For example, shaft 112 can also be used to anchor another part (e.g., a downstream assembly) of the implant within a ventricle downstream of the valve. For example, for implementations in which the implant is (or includes) an artificial chorda tendinea, distal portion 112a of shaft 112 can be used to house a ventricular anchor that is used to anchor the artificial chordae tendineae to the ventricle. In some implementations, distal portion 112a can additionally be used to anchor the ventricular anchor to the ventricle. In some implementations, distal portion 112a can be telescopic, e.g., to facilitate advancement through the ventricle.

In some implementations, tool 110 can be modified, mutatis mutandis, to include one or more features of the delivery tool(s) described in International Patent Application Publication WO 2022/101817 to Tennenbaum et al., and which is incorporated herein by reference. Similarly, tool 110, or a variant thereof, can be used to implant one or more of the implants (and/or the upstream assembly thereof) described in WO 2022/101817 to Tennenbaum et al. Similarly, delivery tools described in WO 2022/101817 to Tennenbaum et al. can be modified to include features of leaflet-grasping tool 110, mutatis mutandis. For example, the upstream and/or downstream support and/or the anchor-driving components of tool 110 can be used in place of the clasp and/or the anchor driver(s) of a delivery tool described in WO 2022/101817 to Tennenbaum et al.

Additionally, or alternatively, shaft 112 and/or tool 110 can be a component of a delivery assembly (e.g., a delivery tool thereof) (not shown) that can be used to anchor a leaflet repair device (e.g., a leaflet-augmentation device such as a flexible wing adapted to improve coaptation of the leaflets) to leaflet 7. For example, a variant of system 100 can be used, mutatis mutandis, to implant an implant described in International Patent Application Publication WO 2022/006087 to Chau et al., which is incorporated herein by reference. Similarly, delivery tools described in WO 2022/006087 to Chau et al. can be modified to include features of leaflet-grasping tool 110, mutatis mutandis.

The approaches to leaflet-grasping described herein could also be used to attach an implant, e.g., an edge-to-edge repair implant, to one or more leaflets of a valve. That is, rather than a tool that is used to deliver and/or implant an implant to a leaflet (e.g., such as tool 110), the leaflet-grasping components described herein can be components of the implant (e.g., serving to anchor the implant). For example, the implant itself (e.g., an edge-to-edge implant, a leaflet clip, a prosthetic spacer, and/or an anchor thereof) can comprise an upstream support and/or a downstream support, at least one of the supports being lengthenable within the heart by increasing the length of that support that is exposed within the heart. For example, a variant of system 100 can be used, mutatis mutandis, to attach one or more of the valve repair devices (or components thereof) described in International Patent Application PCT/US2019/055320, published as WO 2020/076898, which is incorporated herein by reference. For such implementations, once the implant is implanted at the leaflet, e.g., once delivery tool(s) used to deliver the implant to the heart are withdrawn from the heart, the leaflet can remain sandwiched between the upstream support and the downstream support of the implant (e.g., the upstream support and downstream support can remain as anchors of the implant).

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

What is claimed is:

1. A system for use at a heart of a subject, the system comprising a leaflet-grasping tool, the leaflet-grasping tool comprising:
a shaft, having a distal portion that is transluminally advanceable to a valve of the heart;
a first wire, advanceable out of the distal portion such that the first wire extends laterally away from the shaft to form an upstream support;
a second wire, advanceable out of the distal portion such that the second wire extends laterally away from the shaft to form a downstream support; and
a control mechanism, attached to the second wire and adapted to deflect the downstream support with respect to the shaft.

2. The system according to claim 1, wherein the control mechanism is configured to constrain the downstream support in a deflected state during advancement of the second wire out of the distal portion.

3. The system according to claim 2, wherein the leaflet-grasping tool is configured to dynamically limit lateral extension of the first wire out of the distal portion according to lateral extension of the second wire out of the distal portion.

4. The system according to claim 2, wherein the leaflet-grasping tool is configured to dynamically limit lateral extension of the second wire out of the distal portion according to lateral extension of the first wire out of the distal portion.

5. The system according to claim 1, wherein the control mechanism is adapted to push distally on the downstream support in order to deflect the downstream support.

6. The system according to claim 1, wherein the leaflet-grasping tool is adapted to grasp a leaflet of the valve by sandwiching a portion of the leaflet between the upstream support and the downstream support by (i) pressing the upstream support against an atrial surface of the leaflet, and (ii) pressing the downstream support against a ventricular surface of the leaflet.

7. The system according to claim 6, wherein the first wire defines a frame, and the upstream support comprises a sheet attached to the frame, such that the sheet presses against the atrial surface of the leaflet.

8. The system according to claim 6, wherein the second wire defines a frame, and the downstream support comprises a sheet attached to the frame, such that the sheet presses against the ventricular surface of the leaflet.

9. The system according to claim 6, wherein:
the system further comprises an implant, and
the leaflet-grasping tool is adapted to secure the implant to the leaflet while grasping the leaflet.

10. The system according to claim 6, wherein the leaflet-grasping tool further comprises a control rod, attached to the first wire, and adapted to deflect the upstream support with respect to the shaft.

11. The system according to claim 10, wherein the control rod is configured to hold the upstream support deflected away from the leaflet during advancement of the first wire out of the distal portion.

12. The system according to claim 11, wherein the control rod is configured to facilitate the sandwiching by deflecting the upstream support toward the downstream support.

13. A system for use at a heart of a subject, the system comprising a leaflet-grasping tool, the leaflet-grasping tool comprising:
a shaft, having a distal portion that is transluminally advanceable to a valve of the heart;
a first wire, advanceable out of the distal portion such that the first wire extends laterally away from the shaft to form an upstream support;
a second wire, advanceable out of the distal portion such that the second wire extends laterally away from the shaft to form a downstream support; and
a control rod, attached to the first wire and adapted to deflect the upstream support with respect to the shaft
wherein the leaflet-grasping tool is configured to dynamically limit lateral extension of the second wire out of the distal portion according to lateral extension of the first wire out of the distal portion.

14. The system according to claim 13, wherein the leaflet-grasping tool comprises a controller, coupled to a proximal portion of the shaft, the controller adapted to adjust the lateral extension of the first wire away from the shaft.

15. The system according to claim 14, wherein the controller is adapted to adjust the lateral extension of the first wire away from the shaft between a plurality of discrete extensions.

16. The system according to claim 14, wherein the controller is adapted to adjust the lateral extension of the first wire away from the shaft over a continuum of extensions.

17. The system according to claim 14, wherein the controller is adapted to control the deflection of the upstream support with respect to the shaft, independently of the lateral extension of the first wire away from the shaft.

18. The system according to claim 14, wherein the controller is adapted to adjust the lateral extension of the second wire away from the shaft independently of the lateral extension of the first wire away from the shaft.

19. The system according to claim 13, wherein the leaflet-grasping tool is adapted to grasp a leaflet of the valve by sandwiching a portion of the leaflet between the upstream support and the downstream support by (i) pressing the upstream support against an atrial surface of the leaflet, and (ii) pressing the downstream support against a ventricular surface of the leaflet.

20. The system according to claim 19, wherein:
the first wire defines a frame, and the upstream support comprises a sheet attached to the frame, such that the sheet presses against the atrial surface of the leaflet, and
the second wire defines a frame, and the downstream support comprises a sheet attached to the frame, such that the sheet presses against the ventricular surface of the leaflet.
